(19) United States
(12) Patent Application Publication
(10) Pub. No.: US 2024/0197949 A1
(43) Pub. Date: Jun. 20, 2024

(54) NEGATIVE ION BASED CONTINUOUUS DISINFECTION SYSTEM

(71) Applicant: STRL BIOSYSTEMS PRIVATE LIIMITED, Telangana (IN)

(72) Inventors: Bhanuchandra Marepally, Telangana (IN); Lakshmisantosha Valliakella, Telangana (IN); Ravi Teja Avasarala, Telangana (IN)

(73) Assignee: STRL BIOSYSTEMS PRIVATE LIIMITED, Telangana (IN)

(21) Appl. No.: 18/288,091

(22) PCT Filed: Apr. 26, 2022
(86) PCT No.: PCT/IB2022/053837

§ 371 Date: Oct. 24, 2023

(30) Foreign Application Priority Data
Apr. 26, 2021 (IN) 202141019111

(51) Int. Cl. A61L 9/20; A61L 9/22; B01D 53/00; B01D 53/86; B01D 53/88; B01J 23/06; B01J 23/34; B01J 35/39; B01J 35/58

(52) U.S. Cl. CPC A61L 9/205; A61L 9/22; B01D 53/007; B01D 53/8675; B01D 53/8687; B01D 53/8696; B01D 53/885; B01J 23/06; B01J 23/34; B01J 35/39; B01J 35/58; A61L 2209/111; A61L 2209/12; A61L 2209/14; B01D 2255/20707; B01D 2255/2073; B01D 2255/20792; B01D 2255/40; B01D 2255/802; B01D 2257/106; B01D 2257/70; B01D 2258/06; B01D 2259/804

(57) ABSTRACT

The present invention relates to a continuous negative ion based indoor air and surface disinfection system (100) incorporating a nano-composite photo-catalyst that will activate photo-catalytic oxidation at a short-wavelength ultraviolet (UV) light energy. The continuous negative ion based indoor air and surface disinfection system incorporates at least one ultraviolet (UV) light source (104); a photo-catalytic oxidation material (106) which leads to production of oxygen and hydroxyl free radicals when illuminated with ultraviolet (UV) light source in the presence of water vapors. The photo-catalytic oxidation material (106) consisting of a mesh with TiO₂ nano-spindled structures (202) placed over aluminium foil (204) coated with a composite mixture of ZnO—TiO₂.

### FIELD OF THE INVENTION

The present invention relates to a continuous disinfection system. More specifically, the invention provides a method to design and fabricate continuous negative ion and reactive oxygen species based air and surface disinfection system with nano-catalytic UV activation.

### BACKGROUND OF THE INVENTION

Indoor Air quality (IAQ) refers to the air quality within and around buildings and structures. The major indoor pollutants include Asbestos, Biological Pollutants, Carbon Monoxide (CO), Formaldehyde/Pressed Wood Products, Lead (Pb), Nitrogen Dioxide (NO2), Pesticides, Indoor Particulate Matter, Secondhand Smoke/Environmental Tobacco Smoke, Volatile Organic Compounds (VOCs) etc. The exposure to biological contaminants is one of the most significant problems within homes and the workplace today.

The major sources of the biological contaminants include pollens, which originate from plants, viruses, which are transmitted by people and animals, bacteria, which are carried by people, animals, and soil and plant debris, household pets, which are sources of saliva and animal dander (skin flakes) droppings and body parts from cockroaches, rodents and other pests or insects viruses and bacteria and contaminated central air handling systems can become breeding grounds for mold, mildew and other sources of biological contaminants and can then distribute these contaminants through the home. Many of these biological contaminants are small enough to be inhaled, especially in the enclosure of the four walls.

The inhalation of biological contaminants becomes alarming in areas wherein sanitation and sanitization are paramount. Hospitals need a pathogen and particle-free environment, e.g. in Operating Rooms, ICUs and environments for immune suppressed patients as well as for patients with serious allergies. Each year, infectious diseases cause millions of deaths, many of the most common infectious pathogens are spread by droplets or aerosols caused by cough, sneeze, vomiting etc. The knowledge of aerosol transmission mechanisms is limited for most pathogens, although spread by air is an important transmission route for many pathogens including viruses including COVID 19.

Further Healthcare-associated infections (HAI) especially outbreaks of multi-drug-resistant organisms within hospitals are recognized as a major contributor to morbidity and mortality of hospitalized patients. The environmental contaminants of healthcare spaces include skin squames, microorganisms and dust. Further, hospital environments could be source of outbreaks of resistant organisms, and experts suggest indirect transmission via the environment to be as likely as direct person-to-person transmission. Hence, it is equally important for healthcare spaces to take care of removal and dilution of airborne microorganisms, as well as surface contaminants continuously.

Several technologies have proven antibacterial efficacy and these include hydrogen peroxide vapor, ultraviolet (UV) light decontamination, and copper- and silver-coated surfaces. However, their use is limited by high cost or high risk of recontamination. Therefore, there is an urgent need for simple, portable and sensitive devices to eliminate microbes from air and surfaces.

TiO₂ has a band gap in the range of 3.2-3.35 eV depending on the phase of the material. This bandgap signifies light wavelength in the range of 370 nm to 390 nm (near Ultraviolet-UV band) is necessary for excitation of valence band electrons to conduction band and thus, creating the electron (e−)-hole (h+) pairs. These e−-h+ pairs react with the oxygen (O₂) and water vapour (H₂O) present in the air to generate negative ions such as super-oxide ions (O₂₋) and hydroxyl ions (OH*). These negative ions as highly reactive and energetic, which interact with the microbes in the air to destroy their structure at a cellular level. In other words, these negative ions react with Carbon molecular complexes to oxidise them to CO₂ and H₂O. UV—C based light falls in the band range of 100 nm to 280 nm, which is quite energetic than the band gap energy of the TiO₂ material, thus providing with sufficient energy to generate negative ions. In addition, UV-C light has sufficient energy to directly affect the structural integrity of microbe's deoxyribonucleic acid (DNA) that have a band gap in the range of 3.2-4 eV (about >300 nm) much higher than the UV-C light band. Therefore, UV-C has direct effect on the DNA due to its highly ionizing energy. The UV-C photons create an excitation in the base pairs of the DNA thus forming blockades to the replication and self-copying of the microbes. Although UV-C plays a major role in reduction of microbes, it also has negative effects on the human cells and biology. Therefore, UV-C light leakage is a serious concern in most of the UV based air filtrations devices. Microbes ranging from virus to bacteria lie in the range of few 100's of nanometres to a few 10's of microns in size, this makes them very hard to detect with simple instruments and requires complex, precise and expensive equipment to even detect. Current methodizes mostly involve incubating and colonizing, to make the microbial colony into a sizeable area that can be detected, but this consumes valuable time, resources and gives the results of the past state. There is a dire need for an early detection module for the microbes that is economical and simple.

### OBJECT(S) OF THE INVENTION

Accordingly, to overcome the drawbacks of the prior art the main object of the present invention is to provide a continuous negative ion based indoor air and surface disinfection system with nano-catalytic UV activation.

Another object of the present invention is to provide a continuous negative ion based indoor air and surface disinfection system with real time microbial detection and air quality measuring device module.

Yet another object of the present invention is to provide a continuous negative ion and reactive oxygen species based indoor air and surface disinfection system with IOT monitoring and control methods.

Yet another object of the present invention is to provide a continuous negative ion and reactive oxygen species based indoor air and surface disinfection system with UV light leak safety and ozone decomposition features.

Yet another object of the present invention is to provide a continuous negative ion based indoor air and surface disinfection system which can continuously generate negative ions and reactive oxygen species at a high rate that damage the structural integrity of the microbes at a cellular level.

Yet another object of the present invention is to provide a simple and cost effective continuous negative ion and reactive oxygen species based indoor air and surface disinfection system, which can be customized for ready use by hospitals, intensive care units (ICU), sterile rooms, elevators, and common households.

Yet another object of the present invention is to provide a simple and cost effective continuous negative ion based indoor air and surface disinfection system, which can be customized into portable and compact modules to be used in small enclosed space such as a car, an air conditioner, etc. with minimal energy usage.

### SUMMARY OF THE INVENTION

In carrying out the above objects of the present invention, in one embodiment of present invention provides a continuous negative ion based indoor air and surface disinfection system incorporating a nano-composite photo-catalyst that will activate photo-catalytic oxidation on exposure to short-wavelength ultraviolet (UV) light.

In another embodiment of the present invention, a continuous negative ion based indoor air and surface disinfection system coupled within a casing having an air inlet and an air outlet. The continuous negative ion based indoor air and surface disinfection system incorporates at least one ultraviolet (UV) light source; a photo-catalytic oxidation material which leads to production of oxygen and hydroxyl free radicals when illuminated with ultraviolet (UV) light source in the presence of humidity; a negative ion blower unit, an exit filter to minimize the residual UV photons leaking to the external environment, plurality of sensors to perform real time microbial detection along with air quality measuring and an IOT module application that performs monitoring and controlling operation.

In yet another embodiment of the present invention, the photo-catalytic oxidation material is either in mesh or foil form and are placed on all internal faces on the provided casing in order to improve the capacity and efficiency of photo-catalytic oxidation. In present invention, the photo-catalytic oxidation material consists of a mesh with TiO₂ nano-spindled structures placed over aluminium foil coated with a composite mixture of ZnO—TiO₂.

In yet another embodiment of the present invention, the exit filter is a fiberglass filter coated with ZnO/MnO₂ composite mixture. The exit filter functions as a photo-down convertor to minimize the residual UV photons leaking to the external environment and as a catalyst to convert back the harmful ozone to oxygen.

In yet another embodiment of the present invention, the invention provides a method for treating waste gases in the air by using the above said filtration system through irradiating UV light on the surface of photo-catalytic mesh or foil materials to generate free electron and electron hole pairs which can decompose organic (carbon based) impurities in the air into harmless products.

In yet another embodiment of the present invention, a method for continuously disinfecting indoor air and surfaces using the above mentioned indoor air and surface disinfection system, comprising:
circulating air of an indoor space through the casing;
receiving real-time air quality parameters at inlet on IOT module application detected by inbuilt sensors;
circulating the air using a negative ion blower unit placed within or adjacent to the casing;
exposing the air to UV light source and photo-catalytic oxidation material within the casing;
generation of reactive oxygen species (ROS) ions and negative ions through water vapour present in the air;
receiving real-time air quality parameters at outlet on IOT module application detected by inbuilt sensors; and
exhausting the reactive oxygen species (ROS) ions and negative ions into the external space or room or environment via the negative ion blower unit going through an exit filter; wherein,
the photo-catalytic oxidation material is either in mesh or foil form and are placed on all internal faces on the provided casing in order to improve the capacity and efficiency of photo-catalytic oxidation; and
the exit filter minimizes the residual UV photons leaking to the external environment and act as a catalyst to convert back the harmful ozone to oxygen.

### BRIEF DESCRIPTION OF THE DRAWING

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 is a perspective view of the negative ion based indoor air and surface disinfection system according to the embodiments of the present invention;

FIG. 2 is an exploded perspective view of the negative ion based indoor air and surface disinfection system according to the embodiments of the present invention;

FIG. 3 is a schematic illustration showing structure of the photo-catalytic oxidation material according to the embodiments of the present invention;

FIG. 4 is a flow chart describing the sequence involved in optimizing performance of the negative ion based indoor air and surface disinfection system according to the embodiments of the present invention;

FIGS. 5a and 5b are images showing the variation in the colony growth of the microbes with exposure to the negative ions and reactive oxygen species (ROS) ions of the filtration system and without exposure to the filtration system;

FIG. 6 represents a graph to illustrate the absorption spectra of TiO₂ nano-spindled photo-catalytic oxidation material according to the embodiments of the present invention in comparison to standard TiO₂ composition; and

FIG. 7a, 7b and 7c shows validation results of the negative ion based air filtration system in the hospital setting having high bacterial load in the environment.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

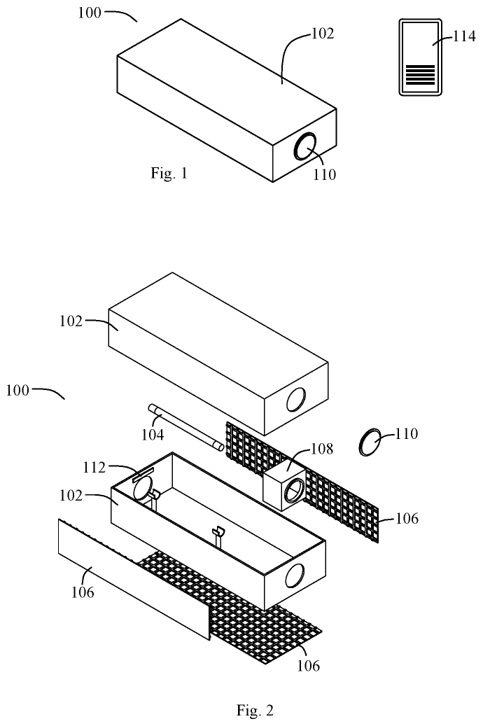

Referring to FIG. 1, shows a perspective view of the continuous negative ion based indoor air and surface disinfection system 100 according to the embodiments of the present invention. The system 100 coupled within a casing 102 having an air inlet and an air outlet. The casing 100 is made up of corrosion resistance material such as, but not limited to plastic, stainless steel, aluminium, etc.

Referring to FIG. 2, shows an exploded perspective view of the continuous negative ion based indoor air and surface disinfection system 100 according to the embodiments of the present invention. The continuous negative ion based indoor air and surface disinfection system 100 incorporates at least one ultraviolet (UV) light source 104. The ultraviolet (UV) light source 104 is preferably transmitting UV light of 365 nm. Plurality of photo-catalytic oxidation material 106 meshes or foil and are placed on all internal faces on the provided casing 102 in order to improve the capacity and efficiency of photo-catalytic oxidation.

The photo-catalytic oxidation material 106 leads to production of oxygen and hydroxyl free radicals when illuminated with ultraviolet (UV) light source 104 in the presence of water vapor present in incoming air. A negative ion blower unit 108 will be placed on the outlet side of casing 102 to pump out the negative ion and reactive oxygen species (ROS) ions into the external space or room or environment through an exit filter 110. The negative ion blower unit 108 is any standard exhaust fan such as, but not limited to axial fans, centrifugal fans, squirrel cage fans, etc.

The exit filter 110 is provided to minimize the residual UV photons leaking to the external environment and act as a catalyst to convert back the harmful ozone to oxygen. The exit filter 110 is a fiberglass filter coated with ZnO/MnO₂ composite mixture.

Plurality of sensors 112 are provided at the inlet and outlet of casing 102 to perform real time microbial detection along with air quality measuring. In an embodiment, the sensor 112 comprises of different sensors such as, but not limited to Wi-Fi module, ozone sensor, particulate matter sensor, carbon dioxide sensor, carbon monoxide sensor, sulfur dioxide sensor, nitrous oxide sensor, etc. An IOT module application 114 is also provided that performs monitoring and controlling operation. The IOT module application 114 can be installed and operated through any network based device such as, but not limited to mobile, tablet, etc. The IOT module application 114 is specifically designed with long range (LoRa) technology that creates a channel for real-time, remote monitoring and a central control via an app.

In another embodiment of the present invention, the invention provides a method for treating waste gases in the air by using the above said filtration system through irradiating UV light on the surface of photo-catalytic mesh or foil materials to generate free electron and electron hole pairs which can decompose organic (carbon based) impurities in the air into harmless products.

Following are the reactions for the generation of the electron-hole pairs, ROS ions and negative ions:

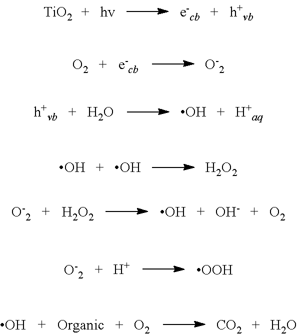

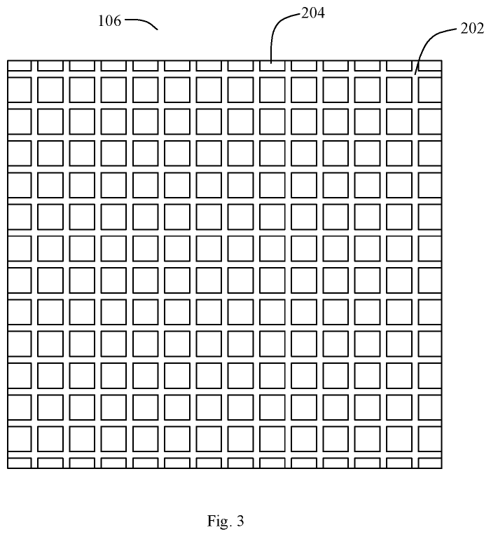

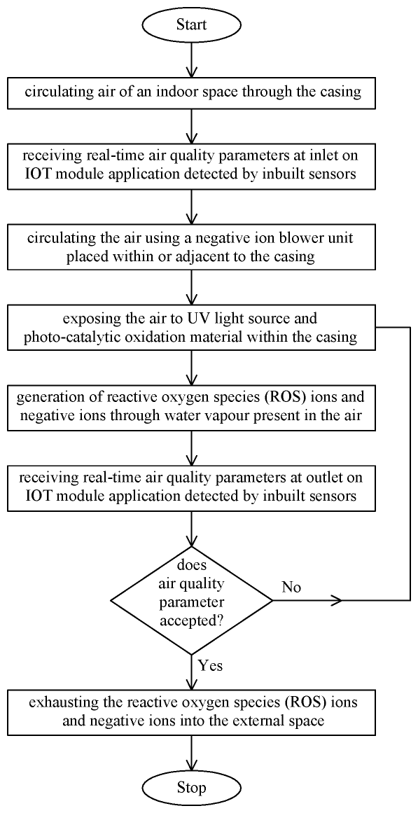

Referring to FIG. 3 is a schematic illustration showing structure of the photo-catalytic oxidation material 106 according to the embodiments of the present invention. In present invention, the photo-catalytic oxidation material 106 comprises of a mesh with TiO₂ nano-spindled structures 202 placed over aluminium foil 204 coated with a composite mixture of ZnO—TiO₂.

In order to prepare TiO₂ nano-spindled structures 202 mesh, following items are required—titanium (Ti) mesh (100-micrometre thickness), iron (Fe) foil, ammonium flouride (NH₄F), hydrofluoric acid (HF), hydrochloric acid (HCl), iso-propanol and de-ionized (DI) water. Firstly, the Titanium (Ti) mesh and Iron (Fe) foil were rinsed with DI water and mechanically polished using polishing sandpaper (p400) and rinsed again with DI water. The polished mesh and foil are rinsed and cleaned ultrasonically, with Iso-Propanol and DI water, successively to form the basic electrodes of the system. The clean electrodes are placed in an electro-deposition setup with electrolyte as 0.2 M NH₄F and 0.1 M HF aqueous solution. The titanium (Ti) mesh forms the working electrode and the iron (Fe) foil forms the cathode. The constant potential difference between the electrodes was maintained at 5V and the distance between the two electrodes at 1 cm. TiO₂ nano-spindle like structures were grown on titanium (Ti) mesh substrate for 2 hours. After completion of this, as a next step, the catalytic electrode is cleaned with DI water for multiple times and later it is rinsed with 0.1 M HCl aqueous solution for three times.

In order to prepare composite mixture of ZnO—TiO₂ for aluminium foil 204, commercially available oxides of Zn and Ti are mixed in the ratio of 1:4 by weight in DI water and then spray coated on to aluminium foil 204 and left to dry for 24 hours. This coating serves as a secondary layer of photo-catalytic oxidation material below the mesh photo-catalytic oxidation material.

Referring to FIG. 4, which presents a flow chart describing the sequence involved in optimizing performance of the negative ion based indoor air and surface disinfection system according to the embodiments of the present invention. First, circulating air of an indoor space through the casing and receiving real-time air quality parameters at inlet on IOT module application detected by inbuilt sensors. After receiving the air quality parameters at inlet the air will be circulated using a negative ion blower unit placed within or adjacent to the casing. Inside the casing, the air will be exposed to UV light source and photo-catalytic oxidation material. Now, this exposure leads to generation of reactive oxygen species (ROS) ions and negative ions through water vapour present in the air. Since reaction take place inside the casing, a real-time air quality parameters from outlet sensor will be received on IOT module application and if the limits of impurities are permissible the negative ion blower unit will exhaust the reactive oxygen species (ROS) ions and negative ions into the external space or room or environment through an exit filter.

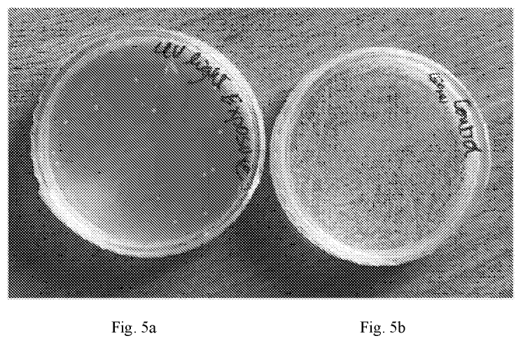

Referring to FIGS. 5a and 5b are images showing the variation in the colony growth of the microbes on nutrient agar plates with exposure to the negative ions and reactive oxygen species (ROS) ions of the filtration system as depicted in FIG. 5a and without exposure to the filtration system as depicted in FIG. 5b. It is evident that the growth of the microbes is significantly reduced in the environment with the exposure to the negative ions and reactive oxygen species (ROS) ions of the filtration system.

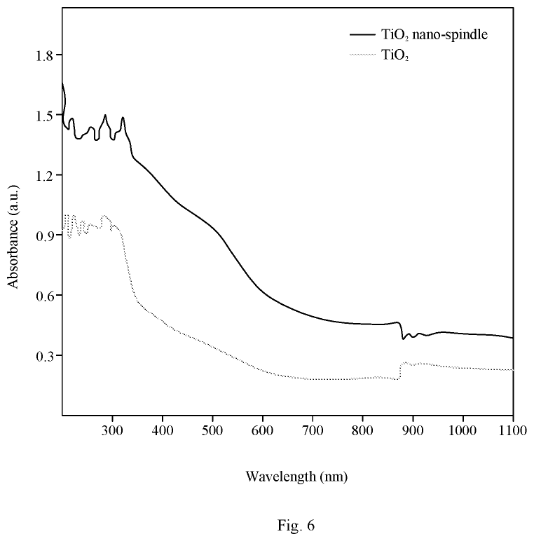

Referring to FIG. 6 represents a graph to illustrate the absorption spectra of TiO₂ nano-spindled photo-catalytic oxidation material according to the embodiments of the present invention in comparison to standard TiO₂ composition. It is evident through the plot that TiO₂ nano-spindled photo-catalytic oxidation material has better absorbance throughout and especially in UV light (200-350 nm). Thus, TiO₂ nano-spindled photo-catalytic oxidation material can continuously generate negative ions at a high rate that damage the structural integrity of the microbes at a cellular level.

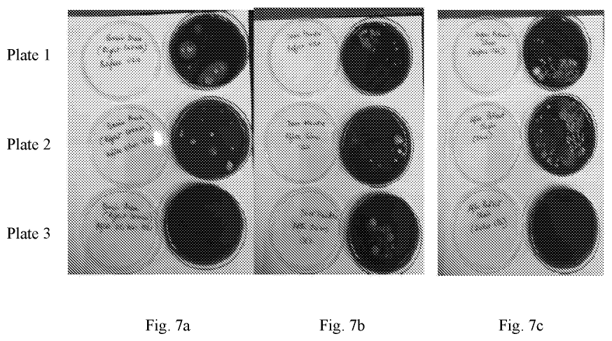

Referring to FIG. 7a, 7b and 7c shows validation results of the negative ion based air filtration system in the hospital setting having high bacterial load in the environment. In order to validate results of the negative ion based air filtration system, two different experimental setups were considered for a wound dressing room in a hospital.

Set up I: The negative ion based air filtration system was placed in the wound dressing room and no human movement was allowed in the room for 72 hours. This activity was done in the evening post OPD hours.

Set up II: The negative ion based air filtration system was placed in the wound dressing room and all human movement was allowed in the room for 72 hours. This activity was done during the OPD hours.

Analysis was done in both set ups I & II before and after the use negative ion based air filtration system.

The following techniques were used for validation—

### Exposure Plate Method—

Nutrient agar petri dishes/plates were prepared and sterilized as per general microbiology protocol. These sterilized plates were placed in the four corners of the room and a fifth plate near the entrance. These plates were exposed to the environment for 30 minutes followed by closing the plates and incubating the plates at 37° C. for 5 days in aerobic conditions. The temperature is the ambient temperature for most of growth of most of the contaminants found in such environment.

### Environmental Swab Culture—

Swab cultures were taken from random places e.g. patient chair, instrument tray, door handle etc. The swab culture was swiped on sterile nutrient agar plate and incubated the plates at 37° C. for 5 days in aerobic conditions. The temperature is the ambient temperature for most of growth of most of the contaminants found in such environment.

The instrument was validated using microbiology air and swab culture done at the following time points: Before the use of negative ion based air filtration system—samples collected at 9 am; after 6 hours use of negative ion based air filtration system— samples collected at 9 am (6 hrs use then switched off overnight, in order to see if organisms re-grow etc); and after 24 hours use of the negative ion based air filtration system.

The assessment of success was measured by 10 Log decrease in microbial growth on culture of the exposure plates and environmental swab cultures done in both Set up I and Set up II.

FIG. 7a shows result of basin area of the wound dressing room. Plate 1 depicts the image of exposure plate kept near the basin area before cleaning of the room in the morning and use of negative ion based air filtration system. The plate was incubated at 37° C. for 72 hours. Microbial growth was seen in plate 1. Plate 2 depicts the image of exposure plate kept near the basin area after 6 hours of use of negative ion based air filtration system. The plate was incubated at 37° C. for 72 hours. Microbial growth was seen in plate 2. Plate 3 depicts the image of exposure plate kept near the basin area (right corner of the room) after continuous use of negative ion based air filtration system for 24 hours. The plate was incubated at 37° C. for 72 hours. No microbial growth is seen in plate 3. This shows that the negative ion based air filtration system worked even after 24 hours of exposure.

FIG. 7b shows result of door handle of wound dressing room. Plate 1 depicts the image of plate 1 with environmental swab taken from the door handle before the cleaning of the room in the morning and use of negative ion based air filtration system. The plate was incubated at 37° C. for 72 hours. Microbial growth is seen in plate 1. Plate 2 depicts the image of plate 2 with environmental swab taken from the door handle after 6 hours of use of negative ion based air filtration system. The plate was incubated at 37° C. for 72 hours. Microbial growth is seen in plate 2. Plate 3 depicts the image of plate with the environmental swab taken from the door handle after 24 hours of use of negative ion based air filtration system. The plate was incubated at 37° C. for 72 hours. Even after 24 hours of use of negative ion based air filtration system the microbial growth is seen. However, the results obtained were expected because the door handle is a region that is repeatedly touched by various people and therefore areas like these always have an exceptionally high microbial load. Such surfaces are therefore recommend to be cleaned directly by pouring or wiping the surface with a disinfectant solution (e.g. Hyphochloride etc).

FIG. 7c shows result of patient chair in wound dressing room. Plate 1 depicts the image of plate with the environmental swab taken from the patient chair before the cleaning of the room in the morning and use of negative ion based air filtration system. The plate was incubated at 37° C. for 72 hours. Microbial growth is seen in plate 1. Plate 2 depicts the image of plate with the environmental swab taken from the patient chair after 6 hours of use of negative ion based air filtration system. The plate was incubated at 37° C. for 72 hours. Microbial growth is seen in plate 2. Plate 3 depicts the image of plate with the environmental swab taken from the patient chair after 24 hours of use of negative ion based air filtration system. The plate was incubated at 37° C. for 72 hours. After 24 hours use NO microbial growth was seen in plate 3. This shows that the negative ion based air filtration system worked even after 24 hours of exposure.

Further, continuous exposure of negative ion based air filtration system airflow for 8 hours a day over 14 days on Albino Mice & Wistar Rats did not produce any adverse or toxic effects for both male and female species. These were confirmed by a continuous exposure test and subsequent analysis of physical, haematological, clinical and histopathological parameters of said mice. Thus, showing that the system (100) is nontoxic unlike other chemical disinfectants.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention.

We claim:

1. A negative ion based air filtration system 100, comprises:
an internally hollow casing 102 having an air inlet and an air outlet;
the casing 102 supporting at least one short-wavelength ultraviolet (UV) light source 104 to activate photo-catalytic oxidation of a nano-composite photo-catalytic oxidation material 106 placed on all internal faces of the casing 102 to generate negative ion and reactive oxygen species (ROS) ions;
a negative ion blower unit 108 placed on the air outlet to pump out the negative ion and reactive oxygen species (ROS) ions;
an exit filter 110 to control the discharge of the UV photons;
plurality of sensors 112 provided at inlet and outlet for performing real time microbial load detection and air quality measurement; and an IOT module application 114 that performs monitoring and controlling operation; wherein,
the photo-catalytic oxidation material 106 comprises of a mesh with TiO₂ nano-spindled structures placed over aluminium foil coated with a composite mixture of ZnO—TiO₂, in the ratio of 1:4; and
the exit filter 110 is a fiberglass filter coated with ZnO/MnO₂ composite mixture.

2. The negative ion based air filtration system 100 as claimed in claim 1, wherein the UV light source is configured to emit UV light at 365 nm.

3. The negative ion based air filtration system 100 as claimed in claim 1, wherein the exit filter act as a catalyst to convert back the harmful ozone to oxygen.

4. The negative ion based air filtration system 100 as claimed in claim 1, wherein the system is used for treating waste gases in the air through irradiating UV light on the surface of photo-catalytic materials to generate free electron and electron hole pairs which decompose organic (carbon based) impurities in the air into harmless products.

5. A method for filtering air using a negative ion based air filtration system 100, the steps comprising of:
allowing air to enter into the negative ion based air filtration system 100 via air inlet having inbuilt sensors for determining the real-time air quality through IOT module application;
exposing the air to UV light source and photo-catalytic oxidation material within the casing;
generating reactive oxygen species (ROS) ions and negative ions through water vapour present in the air; and
exhausting the reactive oxygen species (ROS) ions and negative ions into the external space or room or environment via an air outlet of the negative ion blower unit going through an exit filter while receiving real-time air quality parameters at outlet on IOT module application detected by inbuilt sensors; wherein,
the photo-catalytic oxidation material 106 comprises of a mesh with TiO₂ nano-spindled structures placed over aluminium foil coated with a composite mixture of ZnO—TiO₂ in the ratio of 1:4 that produce oxygen and hydroxyl free radicals when illuminated with said ultraviolet light in the presence of moisture in the incoming air; and
the exit filter 110 is a fiberglass filter coated with ZnO/MnO₂ composite mixture.
